(19) United States
(12) Patent Application Publication
(10) Pub. No.: US 2026/0198891 A1
(43) Pub. Date: Jul. 16, 2026

(54) ULTRASOUND IMAGING CATHETER WITH RECOIL COMPENSATION

(71) Applicant: Siemens Medical Solutions USA, Inc., Malvern, PA (US)

(72) Inventors: Lex Garbini, Renton, WA (US); Estelle Camus, Erlangen (DE); Fong Ming Hooi, Bellevue, WA (US); Vasant Salgaonkar, Sunnyvale, CA (US); Wilko Wilkening, Erlangen (DE)

(21) Appl. No.: 19/018,566

(22) Filed: Jan. 13, 2025

(51) Int. Cl. A61B 8/12; A61B 8/00

(52) U.S. Cl. CPC A61B 8/12; A61B 8/4483; A61B 8/54; A61B 8/58

(57) ABSTRACT

For catheter-based ultrasound probes, a compensator is included as part of the imaging catheter. The compensator counters the thrust or other motion of the imaging catheter, physically stabilizing the imaging catheter during imaging of the patient. Recoil compensation may result in more accurate and/or less blurred ultrasound imaging from a catheter.

### BACKGROUND

The present embodiments relate to ultrasound imaging with a catheter, such as an intracardiac echo (ICE) catheter. Ultrasound imaging catheters typically have a one-dimensional (1D) array for imaging a plane from within the patient.

For intracardiac imaging, transient lesion assessment is possible in B-mode, due to tissue heating. However, as the tissue cools, differentiating the ablated tissue in B-mode is difficult due to the acoustic impedance of ablated and unablated tissue being similar. Persistent lesion-assessment may use elastography, such as Acoustic Radiation Force Impulse (ARFI) imaging. ARFI imaging uses an ultrasound pulse (push pulse) emitted by the array of the imaging catheter to create a force acting on the tissue. The force causes mechanical waves (shear waves) in the tissue that are subsequently registered (tracked) using the imaging capabilities of the imaging catheter.

One difficulty with ARFI imaging is that the array must be close to the area under investigation. To place an array close to the area under investigation requires a highly navigable catheter. A highly navigable catheter has a small diameter, which results in the catheter being affected by the thrust associated with ARFI imaging. For example, transmitting the push pulse creates thrust, causing mechanical motion of the imaging catheter. The motion may be 5 mm or more. Other sources of motion, such as varying pressure from the blood flow (e.g., 5 mm or more) and/or tissue motion resulting from the cardiac and/or breathing cycle, may cause undesired mechanical motion of the imaging catheter for any type of ultrasound imaging or force imaging synchronized with the cycle. The motion of the imaging array and catheter may result in distorted ultrasound images as the array moves between transmit and receive. This motion may interfere with the task of registering (tracking) the shear waves in the tissue for ARFI or interfere with other ultrasound imaging.

### SUMMARY

By way of introduction, the preferred embodiments described below include methods, systems, and improvements for catheter-based ultrasound probes. A compensator is included as part of the imaging catheter. The compensator counters the thrust or other motion of the imaging catheter, physically stabilizing the imaging catheter during imaging of the patient. Recoil compensation may result in more accurate and/or less blurred ultrasound imaging from a catheter.

In a first aspect, an ultrasound imaging catheter is provided. A catheter housing is configured for insertion into a patient. A first one-dimensional array of elements is within the catheter housing and configured for ultrasound imaging of the patient. An acoustic transducer is within the catheter housing. The acoustic transducer faces in a different direction than the first one-dimensional array. The acoustic transducer is configured for recoil compensation due to operation of the first one-dimensional array.

In a second aspect, a method is provided for ultrasound imaging with a catheter probe. A patient is imaged with an imaging array in the catheter probe. The imaging array is subject to motion force during the imaging. The motion force is countered with a physical force applied to the catheter during the imaging.

In a third aspect, an ultrasound imaging catheter is provided. A catheter housing is configured for insertion into a patient. A one-dimensional array of elements is within the catheter housing and configured for ultrasound imaging of the patient. A recoil compensator is configured to apply physical force to limit recoil of catheter housing due to operation of the one-dimensional array.

Any one or more of the aspects or concepts summarized above or in the Illustrative Embodiments below may be used alone or in combination. The aspects or concepts described for one Illustrative Embodiment or aspect may be used in other embodiments or aspects. The aspects or concepts described for a method or system may be used in others of a system, method, computer program, or non-transitory computer readable storage medium.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound system for catheter imaging;

FIG. 2 is a longitudinal cross-sectional view of one embodiment of a part of a catheter with a compensator for recoil;

FIG. 3 is an exploded view of an array and compensator of the catheter of FIG. 2;

FIG. 4 is a flow chart diagram of one embodiment of a method for ultrasound imaging with a catheter-based probe; and

FIG. 5 is a cross-section of an example catheter with a tubular acoustic transducer for recoil compensation.

### DETAILED DESCRIPTION OF THE DRAWINGS AND PRESENTLY PREFERRED EMBODIMENTS

Recoil or motion compensation in a catheter probe (e.g., ICE catheter) allows the probe to remain stationary or move less during imaging. The compensation allows the catheter tip to remain in position by generating an equal but opposite force (e.g., acoustic thrust) to that generated by the ARFI push pulse, other ultrasound imaging transmission, and/or physiological motion. In one approach, the opposite thrust is provided by an acoustic array (or arrays) facing in the opposite direction of the ARFI/imaging array. The compensation limits any global shift in the image data used for tracking shear waves or other ultrasound imaging. The recoil compensation may overcome or limit image-instability due to the deflection of small-diameter catheters that utilize ARFI (or similar forms of elastography) for lesion assessment.

Where ultrasound thrust is used for compensation, the compensation ultrasound pulse may be defocused and/or diffused over the full array area to minimize interference and adhere to the ALARA principle. The B-mode and ARFI imaging are implemented by an array on the front side of the catheter tip. The recoil compensation is implemented by a transducer (e.g., an array or arrays) on the back side of the catheter tip. The back-facing recoil-compensation array can be of a similar form to the front-facing imaging array, or the back facing array can be a simplified and less expensive single-or few-element array. The transmission line to the back-facing array may also be simplified and made less expensive, for example by utilizing only one or two large copper/polyimide strips, instead of 64 individual lines used for a 64-element imaging array.

A control loop may be implemented to optimize the recoil compensation such that the catheter remains steady, even under varying conditions (e.g., anatomical). With or without a control loop, reliable and persistent lesion assessment may allow ablation procedures to be completed with a greater probability of first-time success. Less blurry imaging and/or accurate tissue measurement, assisting in diagnosis or therapy monitoring with ultrasound imaging, may be provided. More accurate placement of ablation electrode and precise control of ablation guided by ultrasound imaging may be provided.

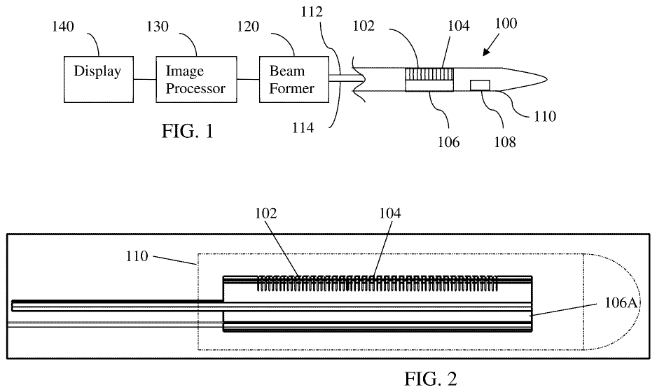

FIG. 1 shows an ultrasound imaging system for medical ultrasound imaging with a catheter probe 100. The ultrasound imaging system is used for diagnosis and/or treatment. The catheter probe 100 includes a recoil or motion compensator for applying force opposite motion force caused by imaging or other sources. The compensator acts to maintain position or limit motion of the catheter probe 100 during ultrasound imaging.

The ultrasound imaging system includes the catheter probe 100 (e.g., array 102 of elements 104 and a housing 110) and an ultrasound scanner (e.g., a beamformer 120, an image processor 130, and a display 140). Additional, different, or fewer components may be provided. For example, the system includes the array 102 in the catheter probe 100 without the beamformer 120, image processor 130, and/or display 140. The transducer array 102 and catheter probe 100 releasably connect with the ultrasound scanner or imaging system. As another example, the beamformer 120 and/or image processor 130 may be integrated on a chip or chips with or adjacent to the array 102.

The conductors 114 connect the array 102 to the beamformer 120 for imaging. The beamformer 120 includes a plurality of channels for generating transmit waveforms and/or receiving signals. Relative delays and/or apodization focus the transmit waveforms or received signals for forming beams. The delays and apodization may be used to diffuse or defocus. The beamformer 120 connects with the conductors 114. The beamformer 120 selects one or more apertures, including one, some, or all the elements 104 of the array 102. Different apertures may be used at separate times. The beamformer 120 may be configured to use one or more channels for transmission of compensation pulses to limit movement or recoil of the array 102. The signals for the compensation pulses are provided on the conductors 114.

The catheter probe 100 includes the housing 110, the array 102 of elements 104, the conductors 114, one or more guide wires 112, compensator 106, and a sensor 108. Additional, different, or fewer components may be provided. For example, a port or tube for inserting and/or withdrawing fluid from the housing 110 is included. As another example, one or more markers (fiducials) for position determination are included. In another example, the sensor 108 is not provided.

The electrical conductors 114 connect the elements 104 of the array 102 to the beamformer 120 and/or connect elements or signal control lines of the compensator 106 to the beamformer 120 or controller (e.g., image processor 130).

Loose wires and/or flexible circuits with traces form the conductors 114. Separate connections are provided for the array 102 and the compensator 106, but multiplexed or shared communications may be used. The conductors 114 are cables, coaxial cables, traces on flexible circuit material, wires, flex circuits (e.g., patterned or deposited traces on flexible circuit material sheet(s)), wire jumpers, combinations thereof, or other now known or later developed conductors. One conductor 114 is provided for each element 104. One conductor 114 is provided for each element or controllable component of the compensator 106. Alternatively, fewer conductors 114 than elements 104 and components may be used, such as for switched apertures, partial beamforming, or multiplexing.

The housing 110 is a sleeve of plastic or other material for insertion into a patient. For example, the housing 110 is formed from Pebax. Other materials, such as other Nylons or biologically neutral (or biocompatible) materials, may be used. The housing 110 is sealed over the array 102 to separate fluids of the patient from the interior of the probe 100.

The housing 110 is configured for insertion into a patient. In general, the housing 110 is cylindrical in shape, such as a long, thin tube. The housing 110 may be stiff, rigid, flexible, and/or semi-flexible. The housing 110 is shaped and sized to form the insertable portion of the catheter probe 100. In one embodiment, the housing 110 forms an ICE catheter with the array 102. The array 102 may fit within 10 French (3.33 mm), 12.5 French, or another diameter catheter. For lesion assessment with ARFI using an ultrasound imaging catheter, the diameter may be 10 French, 8 French, or smaller. In other intraluminal probes, the housing 110 forms TEE, transurethral probe, or endovaginal probe. The probe 100 and corresponding housing 110 may form a micro-TEE for pediatric applications. Examples herein will be for a catheter, but the array 102 may be used in various other intraluminal probes 100. The probe 100 is for imaging or for therapeutic application, such as being used to apply high intensity focused ultrasound (HIFU). The images assist in diagnosis, catheter or tool guidance, and/or therapy placement.

An imaging array 102 is in or on the catheter probe 100. For example, the array is within the catheter housing 110. The array 102 has a plurality of elements 104, electrodes, and a matching layer. Additional, different, or fewer components may be provided, such as a backing block. For example, two or more matching layers are used. As another example, a semiconductor chip (e.g., application specific integrated circuit) is stacked with the array 102 in the catheter housing 110.

The elements 104 may contain piezoelectric material. Solid, single crystal, or composite piezoelectric materials may be used. Each element is a rectangular solid, cube, or six sided, but other surfaces may be provided. For example, the emitting face of one or more elements 104 is planar but may be concave or convex for elevation focusing or frequency-based directivity. The elements may be merged into or placed against a backing block. Alternatively, a microelectromechanical device, such as a flexible membrane, is used. Any now known or later developed ultrasound transducer may be used.

Any number of elements 104 may be provided, such as eight, thirty-two, sixty-four, one hundred and twenty-eight, or more elements, for the array 102. The elements 104 are adjacent to each other, such as having substantially wavelength or less spacing between the centers of adjacent elements 104.

In one embodiment, the array 102 is a 1D array. The elements 104 are distributed along a straight or curved line to form the 1D array of elements 104. In other embodiments, the array 102 is a 1.5D or 2D array (multi-dimensional). In yet other embodiments, any array having one dimension greater than the width (diameter) of the probe body and another dimension less than the width may be used. For example, a planar imaging array produced as a capacitive micromachined ultrasound transducer (CMUT) where each element is composed of a matrix of micro-elements is used.

In one example, the array 102 has 64 elements at 110-micron pitch, providing an array length of 7.5 mm along azimuth. The elements 104 and corresponding array 102 have a width along elevation of 1.8-2 mm. The array 102 is sized for imaging at a 5 MHz center frequency while fitting within an 8-10 French catheter housing 110. The thickness and spacing of the elements 104 configures the elements 104 and resulting array 102 for ultrasound imaging of a patient at a desired frequency band. The array 102 may be configured for transmission of an ARFI (pushing pulse) and tracking transmissions (e.g., B-mode scanning) at a given frequency range and amplitude range and for receiving return echoes. Other arrays 102 may be used, such as with different numbers of elements, element pitches, array lengths, array widths, frequencies of operation, and/or for fitting within different sized housings 110.

The array 102 has an elevation width less than a diameter or width of the probe housing 110. When the azimuth or longitudinal axis of the array 102 is aligned with the longitudinal axis of the housing 110, the array 102 fits within the housing 110. The azimuth lengths of the array 102 are longer than the diameter or width of the housing 110.

The array 102 is positioned distally from the steering section of the housing 110 of the probe 100. The array 102 is in or near a tip of the catheter housing 110. Other positions may be provided. The array 102 is positioned along the longitudinal axis within the housing 110.

The compensator 106 is a motor, drum, acoustic transducer 106A, pump and nozzle (e.g., fluid jet), and/or another device for applying a directional force to the array 102 and/or catheter housing 110. The compensator 106 is configured by design and/or positioning to apply a physical force that may limit or prevent recoil or movement of the catheter housing 110 and/or array 102 due to operation of the array 102 or patient. The compensator 106 counters the movement force caused by the array 102 or another source (e.g., fluid pressure variation and/or tissue motion from physiological cycle). For countering recoil due to transmission or operation of the array 102, the compensator 106 is a recoil compensator.

As a motor, the compensatory 106 operates a gyroscope or armature to cause physical force for compensation. As a drum, an electric signal is used to cause the drum to shift, such as a diaphragm, causing physical force for compensation. As a pump and nozzle, saline or other fluid is jetted from the catheter 100 to cause physical force for compensation. In another implementation, a mass is moveable within the catheter 100. The mass may be attached to a spring or in a viscous liquid. The motor or an arrangement of coils with magnetic mass moves the mass to compensate.

In one embodiment, the compensator 106 is an acoustic transducer 106A. The acoustic transducer 106A is within the catheter housing 110, such as being stacked with or by the array 102. The acoustic transducer 106A may be spaced from the array 102 longitudinally, such as have part proximal and part distal to the array 102. The acoustic transducer 106A is configured for recoil compensation of recoil due to the operation of the array 102 and/or for compensation of motion form other sources. The acoustic transducer 106A is configured to transmit acoustic energy during transmission of acoustic energy by the first one-dimensional array. The placement and/or control from the beamformer 120 configures the acoustic transducer 106A to transmit the acoustic energy.

Calibration, simulation, or estimation may be used to determine the power or thrust to be used for the transmission of the acoustic energy. By transmitting acoustic energy from the acoustic transducer 106A at a same time as transmissions for imaging (e.g., during a pushing pulse (ARFI) in ARFI imaging) from the array 102, the recoil of the array 102 due to the imaging transmissions may be reduced or eliminated.

The arrangement, size, position, direction, beamformer connection, and/or other characteristic of the acoustic transducer 106A configures the acoustic transducer 106A for compensation. By setting one or more characteristics, the acoustic array is configured to function as the compensator 106.

Since the acoustic transducer 106A is not for imaging, focused transmission is not needed. Focused transmission may be used, such as focusing to a location known to cause less interference (e.g., backscatter) or echoes. Due to less strict focusing requirements, the acoustic transducer 106A may be a single element. In other approaches, the acoustic transducer 106A has 2-5 elements (e.g., fewer than six). Other numbers of elements may be used for the compensator 106.

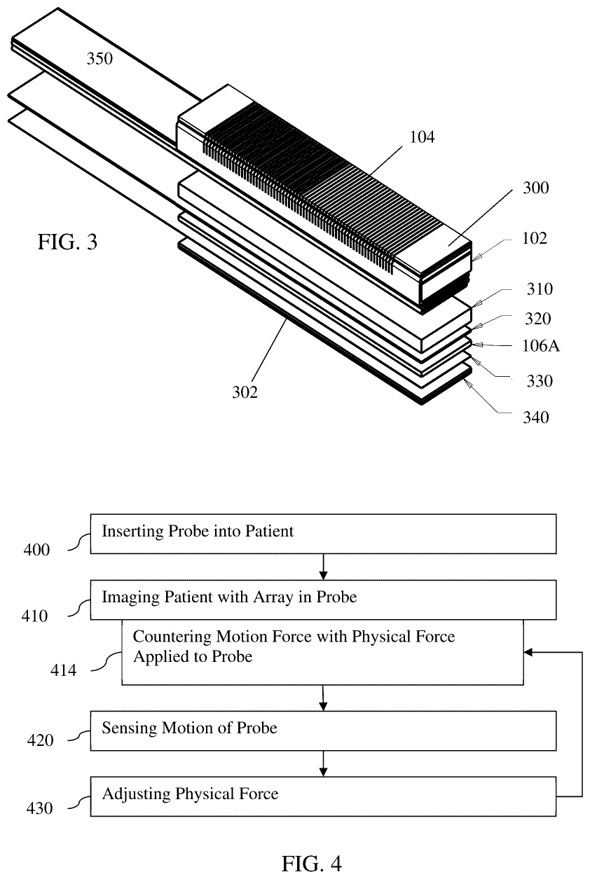

The direction of the acoustic transducer 106A also may configure for compensation. The acoustic transducer 106A faces in a different direction than the array 102, such as facing in an opposite direction. The face of the acoustic transducer 106A is a surface area from which the acoustic energy is primarily emitted. In FIG. 3, the face 300 of the array 102 is positioned close to the imaging area (tissue to be imaged). The acoustic transducer 106A (e.g., compensator 106) has an emitting face 302 substantially parallel with the face 300 but emits in the substantially opposite direction (i.e., away from the tissue to be imaged).

Substantially is used to account for manufacturing tolerance and surface shaping (concave or convex) of the emitting faces 300, 302. In other embodiments, the acoustic array is formed from different elements or arrays facing in different directions, such as +/−30 degrees from perpendicular to a normal from the emitting or transmission face 300 of the array 102. Different arrays or elements may face in different directions. Alternatively, an array of two or more elements is provided for some electronic steering to control the angle of the thrust for recoil compensation.

The size of the acoustic transducer 106A may also configure for compensation. For substantially complete compensation, the acoustic transducer 106A transmits to cause a thrust or force substantially equal to the force causing recoil or motion of the array 102. Substantially accounts for 5% deviation. Where the motion force is from transmission by the array 102, the acoustic transducer 106A transmits to cause a substantially equal thrust from acoustic energy. The amplitude, frequency, and/or duration of the transmitted acoustic energy may be altered to set the thrust. The size of the emitting face 302 also sets the thrust. In one approach, the transmitting face 302 of the acoustic transducer 106A has an area of less than 20 percent different than the area of the transmitting face 300 of the array 102. In the example of FIG. 3, the transmitting faces 300, 302 are substantially equal, where substantially accounts for manufacturing tolerance.

The thickness of the acoustic transducer 106A may also configure for compensation. The frequency band and/or center frequency of the acoustic energy transmitted for compensation establishes the thickness of the acoustic transducer 106A. To avoid interference with imaging, the acoustic transducer 106A transmits at a different frequency than the array 102. Harmonics, such as the second harmonic, may also be avoided. For example, the array 102 transmits at 5 MHz center frequency, and the acoustic transducer 106A transmits at 2 MHz or 12 MHz center frequency. The thickness and/or element spacing of the acoustic transducer 106A is set for transmitting acoustic energy or ultrasound at the desired frequency.

The location may be used to configure. The acoustic transducer 106A is stacked behind or with the imaging array 102. Other locations may be used, such as offsetting the acoustic transducer 106A along a longitudinal direction from the array 102. The location may be used to alter or design the direction of the compensating force. For example, the tip of the catheter 100 may be articulated to the right where the imaging array 102 is looking straight ahead. The operation of the array 102 may case rotation of the catheter beyond the articulation point. The acoustic transducer 106A may be positioned and/or directed to counter the rotation or adjust the articulation point.

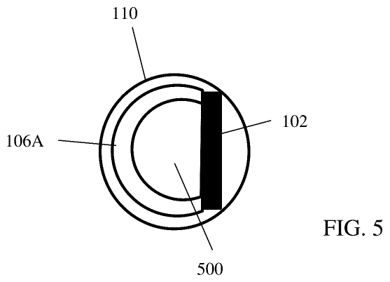

In one implementation, the acoustic transducer 106A has a tubular shape, such as following a shape of the housing 110 over a 45-180 degree arch. FIG. 5 shows an example cross-section of the catheter 100 at the array 102. A backing 500 is between the array 102 and the acoustic transducer 106A. The tubular shape of the acoustic transducer 106A defocuses acoustic pressure for a given amount of energy with a negative pressure low enough to have safe mechanical index (MI). In one example, the tubular acoustic transducer 106A is configured to operate at 8-10 MHz.

FIGS. 2 and 3 show an example arrangement of part of the catheter (i.e., tip or imaging section) with the compensator 106 as an acoustic transducer 106A. The array 102 and acoustic transducer 106A are stacked in the catheter housing 110. The areas of the transmitting faces 300, 302 are substantially equal. The acoustic transducer 106A is aligned with the array 102 but with the transmitting face 302 of the acoustic transducer 106A facing a substantially opposite direction than the emitting face 300 of the array 102. The piezoelectric material of the acoustic transducer 106A is thinner than the piezoelectric material of the array 102 since the acoustic transducer 106A is to transmit acoustic energy at a higher frequency. This also uses less space in the catheter housing 110 than where the acoustic transducer 106A is thicker.

Other structure may be stacked with the array 102 and acoustic transducer 106A. The array 102 as shown includes one or more matching layers, a backing, and flexible circuit material sheets 350 with traces for signal lines and ground. Any now known or later developed array 102 stack may be used.

The array 102 stack is positioned on an optional stiffener 310. The stiffener 310 is positioned between the array 102 and the acoustic transducer 106. The stiffener 310 is formed from nitinol, low magnetic stainless steel, or another material to limit bending of the array 102 and/or acoustic transducer 106A.

The acoustic transducer 106A is sandwiched between or includes two sheets 320, 330 of flexible circuit material, one for providing signal to the acoustic transducer 106 and one for ground. For example, the sheet 330 over the emitting face 302 has a deposited conductor without patterning for ground return. The sheet 320 between the acoustic transducer 106A and the stiffener 310 also has a deposited conductor without patterning for signal where the acoustic transducer 106A is a single element or has multiple elements to receive the same signal. The sheet 320 may have conductor deposited on both sides to provide separate signals to two elements. By having only one or two transmission lines, the cost of patterning traces on the sheet 320 is reduced. In other approaches, multiple traces are patterned on the sheet 320 to provide separate transmit signals to two or more elements of the acoustic transducer 106A.

One or more matching layers 340 are stacked by and/or covering the transducer material, forming the emitting face 302 of the acoustic transducer 106A. The matching layer 340 limits echoes from the fluid or tissue at the boundary adjacent to the acoustic transducer 106A and may improve efficiency of the transducer 106A. The matching layer 340 shifts the acoustic impedance to avoid echoes that may interfere with the imaging by the array 102.

Other arrangements of the acoustic transducer 106A and array 102 may be used. Additional, different, or fewer components may be provided. Any arrangement allowing for imaging by the array 102 and recoil compensation by the acoustic transducer 106A may be used.

In a further approach, an optional sensor 108 is provided in the catheter 100, either distal or proximal or in proximity to the array 102. The sensor 108 is an accelerometer, gyroscope, or position sensor. Other types of sensors to sense motion, acceleration, or the force from the array 102 or patient may be used. The sensor 108 senses the recoil or motion of the array 102. When the compensator 106 is countering, the amount of recoil is desired to be zero. The sensor 108 may sense non-zero recoil and adjust the compensation. The processor 130 controls the beamformer 120 and/or another device (e.g., compensator 106) to alter the magnitude of the compensation in response the sensor 108. The direction may be altered as well where the direction of the motion is determined. Where the sensor 108 and processor 130 operate quickly relative to the compensation, the magnitude of compensation may be adjusted during the compensation. The magnitude may also or instead be adjusted for subsequent compensation.

The ultrasound scanner (e.g., beamformer 120, image processor 130, and/or display 140) is configured for ultrasound imaging. The array 102 is used to form an aperture for a scan plane. The beamformer 120 uses elements 104 of the array 102 to scan an image plane. The beamformer 120 electronically focuses for imaging along a plurality of scan lines. During receive operations, the focus may vary as a function of depth (i.e., dynamic focusing).

The image processor 130 is a detector, filter, processor, application specific integrated circuit, field programmable gate array, digital signal processor, control processor, controller, scan converter, three-dimensional image processor, graphics processing unit, analog circuit, digital circuit, or combinations thereof. The image processor 130 receives beamformed data and generates images on the display 140. The image processor 130 may be a controller for the beamformer 120 and/or compensator 106. The image processor 130 may receive signals from the array 102 and/or sensor 108.

FIG. 4 is a flow chart diagram of one implementation of a method for ultrasound imaging with a catheter or other endoluminal probe. The probe includes compensation to physically stabilize imaging.

The probes of FIGS. 1-3 or another intraluminal probe with a compensator may be used. Any ultrasound system may be used.

Additional, different, or fewer acts may be provided. For example, acts for configuring the ultrasound imaging system and/or acts for diagnosis or treatment are included. As another example, acts 420 and/or 430 are not provided.

The acts are performed in the order shown (top to bottom or numerically) or a different order. For example, acts 414, 420, and/or 430 may be performed simultaneously or during act 410.

In act 400, the catheter probe is inserted into a patient. The probe is inserted into a lumen, such as a blood vessel. For example, the probe is an intracardiac (ICE) catheter inserted into the cardiac system to navigate to the heart. The tip of the probe is positioned for imaging tissue of interest, such as for monitoring ablation or examining a lesion. Guide wires, translation, and/or rotation are used to steer the probe to a position for imaging. The array of the probe is positioned so that a scan plane includes the tissue of interest.

In act 410, the array of the probe is used for imaging. The patient is imaged with an imaging array in the probe, such as in a catheter probe. An ultrasound scanner images in the plane defined by the imaging array. Volume imaging may be provided using a multi-dimensional array, shaped 1D array, or movement of the 1D array.

The array connects with the beamformer to scan the patient. The scan region is scanned with ultrasound, and an ultrasound image or images of tissue and/or fluid of the patient is generated. Any imaging (e.g., B-mode and/or flow or color mode) may be used. In one implementation, elastography (e.g., ARFI) imaging is performed. A pushing pulse with sufficient power to generate a shear or longitudinal wave at a focal region is transmitted from the array. Subsequent B-mode scanning is repetitively performed to track displacements in tissue caused by propagation of the generated shear or longitudinal wave. The elasticity or another tissue characteristic (e.g., shear wave speed) is measured from the tracking. The imaging generates one or more images, such as images representing tissue in a two or three-dimensional region and/or an image showing one or more values of tissue characteristic for a location or multiple locations.

The imaging array is subject to motion force during the imaging. The heart and/or breathing cycle of the patient may result in motion force (velocity and/or acceleration) being applied to the probe. The transmission from the probe may result in motion force (i.e., thrust or velocity) being applied to the probe. The ARFI or pushing pulse may cause more recoil than typical B-mode transmissions. The transmissions for B-mode or another mode may cause motion force (i.e., force causing movement) to the array and probe housing the array. Combinations of force from acoustic transmission and physiological force may be applied to the probe.

In act 414, a compensator counters the motion force with a physical force applied to the probe during the imaging 410. The thrust from imaging and/or from physiological cycles is countered. For example, the thrust from transmission of a pushing pulse is compensated. The counter or compensation limits the motion of the probe and/or array. To stabilize the probe and array during imaging, the compensator compensates for any motion force.

In an imaging example, the imaging causes thrust on the array and probe by transmission of ultrasound. The motion of the array and probe is limited or prevented by transmission or application of an opposite force of the same or substantially same magnitude (i.e., +/−5%). For example, an acoustic force opposite to the thrust from the array is transmitted from another transducer.

In an example countering motion due to heart muscle motion, the countering may be synchronized with the heart cycle. ECG or other heart cycle detection is used to modulate the amplitude and/or activation of the compensator.

This opposite acoustic force has the same or substantially same power or energy. The opposite acoustic force is unfocused, defocused, and/or diffuse. The transmitted pulse may not have a focus, may be purposefully defocused, spread in a diffuse manner, and/or focused to a location that will result in less echo.

Other sources of physical force may be provided to counter the motion force. For example, a jet of fluid, drum, or gyroscopic force may be used.

By application of an opposite or substantially opposite force to the motion force, the array and probe (e.g., tip of the catheter) may be maintained substantially in position relative to the patient during imaging despite the motion force. Substantially maintained accounts for moving less than 10% of the motion that would occur without the compensation. Recoil or other sources of motion are countered entirely or at least partially.

In a further implementation, the stiffness of the catheter is measured using transmissions from the imaging array and/or compensator prior to imaging. Further pulses from the imaging array and/or compensator are used to generate an oscillation in the catheter and motion of the imaging array. The imaging array is then operated when the catheter and imaging array are moving through a zero point in the oscillation. The countering may be applied at that zero point as well.

In act 420, a sensor senses motion of the probe and/or array. The motion is sensed as a change in motion (acceleration) or ongoing motion. Motion of the probe and array is not desired during imaging. The probe and array may move even with compensation or due no compensation being applied. The sensor senses this motion.

In response to sensed motion, the countering of act 414 of the motion may be adjusted in act 430. Where there is no countering of act 414, the adjustment is to apply countering in act 414. Where countering is occurring, the adjustment may be to increase or decrease the countering of act 414. The magnitude and/or direction of the motion may be sensed in act 420. This information is used to adjust. Alternatively, the adjustment is done in steps to find a total change. The sensing of act 420 is ongoing or interleaved with adjustments to find the countering in act 414 with no or substantially no movement of the probe and array.

The ultrasound imaging generated during imaging may be used for feedback. For example, the level of artifact or amount of blur may be used to control the countering of act 414. The relative position of the imaging array to the patient or tissue in the image may be used as an indication of movement for controlling countering in act 414.

Prior knowledge and/or prediction may be used in countering in act 414. Cardiac motion is cyclical, and the pushing pulse will be transmitted at a known time. Using ECG signals, the control may predict or learn when and how much countering force to use given the knowledge of cycle and timing. The pushing pulse may be triggered based on matching of the countering to a particular point in the heart cycle, possibly minimizing the compensation force.

Due to the physical stabilization or limiting of motion, the images from the imaging of act 410 may have less blur or motion artifact. For elastography (e.g., ARFI) imaging, the measured tissue characteristics may be more accurate since measured displacements have less contribution from array movement. The resulting images may provide for more accurate diagnosis or more useful information for making medical decisions.

Listed below are various Illustrative Embodiments. The Illustrative Embodiments summarize different combinations of aspects or features. Other combinations of any of the aspects or features with any other one or more of the aspects or features may be provided. Aspects or features from one type (e.g., method or system) may be used in another type (system or method).

Illustrative Embodiment 1. An ultrasound imaging catheter comprising: a catheter housing configured for insertion into a patient; a first one-dimensional array of elements within the catheter housing, the first one-dimensional array configured for ultrasound imaging of the patient; and an acoustic transducer within the catheter housing, the acoustic transducer facing in a different direction than the first one-dimensional array, wherein the acoustic transducer is configured for recoil compensation due to operation of the first one-dimensional array.

Illustrative Embodiment 2. The ultrasound imaging catheter of Illustrative Embodiment 1, wherein the catheter housing comprises a diameter of ten French or smaller.

Illustrative Embodiment 3. The ultrasound imaging catheter of any of Illustrative Embodiments 1-2, wherein the first one-dimensional array is configured for transmission of an acoustic radiation force impulse and tracking transmissions, and wherein the acoustic transducer is configured to reduce the recoil of the first one-dimensional array in the catheter caused by the transmission of the acoustic radiation force impulse.

Illustrative Embodiment 4. The ultrasound imaging catheter of any of Illustrative Embodiments 1-3, wherein the acoustic transducer is configured to transmit acoustic energy during transmission of acoustic energy by the first one-dimensional array.

Illustrative Embodiment 5. The ultrasound imaging catheter of any of Illustrative Embodiments 1-4, wherein the first one-dimensional array comprises a linear array thirty-two or more of the elements, and wherein the acoustic transducer comprises fewer than six elements.

Illustrative Embodiment 6. The ultrasound imaging catheter of any of Illustrative Embodiments 1-5, wherein the acoustic transducer comprises a transmitting face having an area of less than 20 percent different than an area of a transmitting face of the first one-dimensional array.

Illustrative Embodiment 7. The ultrasound imaging catheter of any of Illustrative Embodiments 1-6, wherein the acoustic transducer is stacked in the catheter with the first one-dimensional array, and wherein a transmitting face of the acoustic transducer faces in a substantially opposite direction as a transmitting face of the first one-dimensional array.

Illustrative Embodiment 8. The ultrasound imaging catheter of any of Illustrative Embodiments 1-7, wherein the acoustic transducer is configured to operate with a center frequency different than a center frequency of the first one-dimensional array.

Illustrative Embodiment 9. The ultrasound imaging catheter of any of Illustrative Embodiments 1-8, further comprising a sensor configured to sense recoil, and a processor configured to adjust the recoil compensation based on the sensed recoil.

Illustrative Embodiment 10. The ultrasound imaging catheter of any of Illustrative Embodiments 1-9, further comprising a stiffener positioned between the first one-dimensional array and the acoustic transducer.

Illustrative Embodiment 11. The ultrasound imaging catheter of any of Illustrative Embodiments 1-10, further comprising a matching layer covering a transmitting face of the acoustic transducer.

Illustrative Embodiment 12. The ultrasound imaging catheter of any of Illustrative Embodiments 1-11, further comprising a sheet of flexible circuit material with only one or two transmission lines connected with the acoustic transducer.

Illustrative Embodiment 13. A method for ultrasound imaging with a catheter probe, the method comprising: imaging a patient with an imaging array in the catheter probe, the imaging array subject to motion force during the imaging; and countering the motion force with a physical force applied to the catheter during the imaging.

Illustrative Embodiment 14. The method of Illustrative Embodiment 13, wherein imaging comprises transmitting a first acoustic pulse from an imaging array in a catheter, the transmission of the first acoustic pulse causing thrust on the imaging array as the motion, and wherein countering comprises compensating for the thrust, the compensation limiting the motion of the catheter.

Illustrative Embodiment 15. The method of Illustrative Embodiment 14, wherein the first acoustic pulse comprises a pushing pulse of acoustic radiation force impulse imaging and wherein compensating comprises transmitting an opposite acoustic force to the thrust.

Illustrative Embodiment 16. The method of any of Illustrative Embodiments 13-15 wherein countering comprises transmitting acoustic energy opposite the motion as the physical force.

Illustrative Embodiment 17. The method of Illustrative Embodiment 16, wherein the acoustic energy comprises unfocused, defocused, and/or diffused acoustic energy, and wherein the motion force is caused by focused transmission of ultrasound.

Illustrative Embodiment 18. The method of any of Illustrative Embodiments 13-17, wherein countering comprises maintaining a tip of the catheter substantially in position relative to a patient during the imaging despite the motion force.

Illustrative Embodiment 19. The method of any of Illustrative Embodiments 13-18, further comprising sensing motion of the catheter probe and adjusting the countering of the motion force based on the sensed motion.

Illustrative Embodiment 20. An ultrasound imaging catheter comprising: a catheter housing configured for insertion into a patient; a one-dimensional array of elements within the catheter housing, the one-dimensional array configured for ultrasound imaging of the patient; and a recoil compensator configured to apply physical force to limit recoil of catheter housing due to operation of the one-dimensional array.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. An ultrasound imaging catheter comprising:
a catheter housing configured for insertion into a patient;
a first one-dimensional array of elements within the catheter housing, the first one-dimensional array configured for ultrasound imaging of the patient; and
an acoustic transducer within the catheter housing, the acoustic transducer facing in a different direction than the first one-dimensional array, wherein the acoustic transducer is configured for recoil compensation due to operation of the first one-dimensional array.

2. The ultrasound imaging catheter of claim 1, wherein the catheter housing comprises a diameter of ten French or smaller.

3. The ultrasound imaging catheter of claim 1, wherein the first one-dimensional array is configured for transmission of an acoustic radiation force impulse and tracking transmissions, and wherein the acoustic transducer is configured to reduce the recoil of the first one-dimensional array in the catheter caused by the transmission of the acoustic radiation force impulse.

4. The ultrasound imaging catheter of claim 1, wherein the acoustic transducer is configured to transmit acoustic energy during transmission of acoustic energy by the first one-dimensional array.

5. The ultrasound imaging catheter of claim 1, wherein the first one-dimensional array comprises a linear array of thirty-two or more of the elements, and wherein the acoustic transducer comprises fewer than six elements.

6. The ultrasound imaging catheter of claim 1, wherein the acoustic transducer comprises a transmitting face having an area of less than 20 percent different than an area of a transmitting face of the first one-dimensional array.

7. The ultrasound imaging catheter of claim 1, wherein the acoustic transducer is stacked in the catheter with the first one-dimensional array, and wherein a transmitting face of the acoustic transducer faces in a substantially opposite direction as a transmitting face of the first one-dimensional array.

8. The ultrasound imaging catheter of claim 1, wherein the acoustic transducer is configured to operate with a center frequency different than a center frequency of the first one-dimensional array.

9. The ultrasound imaging catheter of claim 1, further comprising a sensor configured to sense recoil, and a processor configured to adjust the recoil compensation based on the sensed recoil.

10. The ultrasound imaging catheter of claim 1, further comprising a stiffener positioned between the first one-dimensional array and the acoustic transducer.

11. The ultrasound imaging catheter of claim 1, further comprising a matching layer covering a transmitting face of the acoustic transducer.

12. The ultrasound imaging catheter of claim 1, further comprising a sheet of flexible circuit material with only one or two transmission lines connected with the acoustic transducer.

13. A method for ultrasound imaging with a catheter probe, the method comprising:
imaging a patient with an imaging array in the catheter probe, the imaging comprising transmitting a first focused acoustic pulse from the imaging array, the transmission of the first focused acoustic pulse causing a recoil motion force on the imaging array; and
countering the motion force by transmitting a second acoustic pulse from a separate acoustic transducer within the catheter probe during the imaging, wherein the second acoustic pulse comprises unfocused, defocused, and/or diffused acoustic energy transmitted in a direction opposite to the recoil motion force.

14. (canceled)

15. The method of claim 13, wherein the first focused acoustic pulse comprises a pushing pulse of acoustic radiation force impulse imaging.

16. (canceled)

17. (canceled)

18. The method of claim 13, wherein countering comprises maintaining a tip of the catheter probe substantially in position relative to the patient during the imaging despite the motion force.

19. The method of claim 13, further comprising sensing motion of the catheter probe and adjusting the countering of the motion force based on the sensed motion.

20. An ultrasound imaging catheter comprising:
a catheter housing configured for insertion into a patient;
a one-dimensional array of elements within the catheter housing, the one-dimensional array configured for ultrasound imaging of the patient by transmitting a first acoustic radiation force impulse causing a recoil motion force on the catheter housing; and
a recoil compensator configured to limit recoil motion force of the catheter housing due to operation of the one-dimensional array by transmitting a second acoustic pulse in a direction opposite to the recoil motion force.
